(19) United States
(12) Patent Application Publication
(10) Pub. No.: US 2026/0263182 A1
(43) Pub. Date: Sep. 10, 2026

(54) SECURING PULL WIRES WITHIN A SURGICAL INSTRUMENT HOUSING

(71) Applicant: Auris Health, Inc., Santa Clara, CA (US)

(72) Inventors: Robert Mason GLIDDEN, IV, Emerald Hills, CA (US); Boney Charles MEJIA, Mountain View, CA (US); Adrian HAIRRELL, El Granada, CA (US); Russell PONG, Santa Clara, CA (US)

(73) Assignee: Auris Health, Inc., Santa Clara, CA (US)

(21) Appl. No.: 19/075,211

(22) Filed: Mar. 10, 2025

(51) Int. Cl. A61B 34/00; A61B 34/30

(52) U.S. Cl. CPC A61B 34/70; A61B 34/30; A61B 2034/301; A61B 2034/305

(57) ABSTRACT

A surgical instrument is disclosed that includes a housing, a scope extending from the housing, a terminator arranged within the housing and including a body that provides a wire groove and a shoulder defined in the groove, force isolation tube extending from the scope and being received within the wire groove to the shoulder, pull wire extending within the force isolation tube and through the wire groove, and a pulley arranged within the housing to receive the pull wire from the terminator, wherein rotation of the pulley acts on the pull wire to articulate the scope.

### BACKGROUND

The present disclosure relates to surgical instruments and, more particularly, to pull wire terminators for securing pull wires within a surgical instrument housing.

Pull wires used in various robotic surgical instruments are commonly used to actuate a flexible end of an endoscope or catheter. The pull wires are typically anchored to a pulley or slider within surgical instrument housing or “handle”. To reduce backlash in the system, any slack must be taken out of the pull wires during assembly, which can happen either before or after the pull wire is anchored.

Securing the pull wires within the instrument housing can be a laborious task due to the small size of the pull wires, and can often require a microscope. Accordingly, systems and methods to improve the ease of installing pull wires within the surgical instrument housing are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example medical system that may incorporate the principles of the present disclosure.

FIG. 2 illustrates example medical system components that may be implemented in the medical system of FIG. 1.

FIG. 3 shows an exploded view of an instrument manipulator assembly of FIG. 1 that includes a surgical instrument with an instrument handle and a scope.

FIG. 4 is a cross-sectional end view of the scope of FIG. 3, in accordance with at least one aspect of the present disclosure.

FIG. 5 is an isometric view of the interior of the instrument handle of FIG. 3, in accordance with at least one aspect of the present disclosure.

FIG. 6 is an enlarged isometric view of an example terminator, pull wires, and force isolation tubes disposed within the instrument handle of FIG. 5, in accordance with at least one aspect of the present disclosure.

FIG. 7 is a top-down view of FIG. 6, in accordance with at least one aspect of the present disclosure.

FIGS. 8 and 9 show progressive steps of inserting a pull wire into the terminator of FIG. 6, in accordance with at least one aspect of the present disclosure.

FIG. 10 is an enlarged, isometric view of another portion of the instrument handle of FIG. 3 including a circuit board and a cable, in accordance with at least one aspect of the present disclosure.

FIG. 11 is the isometric view of FIG. 10 with the circuit board omitted to allow viewing of the cable routed through a torturous path, in accordance with at least one aspect of the present disclosure.

### DETAILED DESCRIPTION

The present disclosure relates to surgical systems and, more particularly, to pull wire terminators for securing pull wires within a surgical instrument housing.

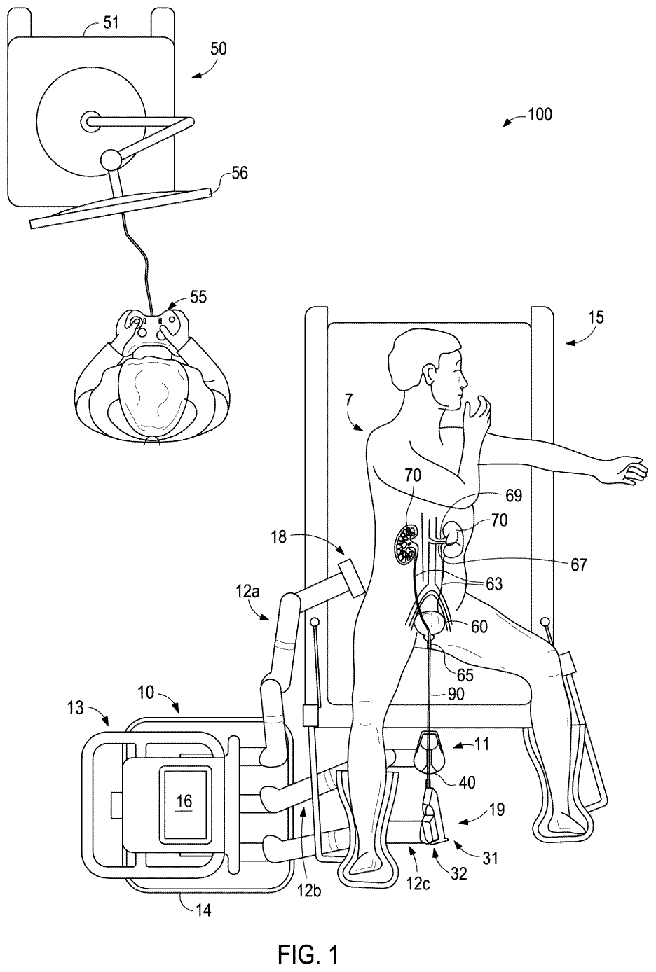

FIG. 1 illustrates an example medical system 100 for performing various medical procedures and that may incorporate aspects of the present disclosure. The medical system 100 may be used for endoscopic (e.g., ureteroscopic) procedures, for example. In some implementations, kidney stone treatment can benefit from the assistance of certain robotic technologies/devices, such as may be similar to those shown in FIG. 1 and described in detail below. Additional information regarding medical system 100 is provided in U.S. Pat. No. 12,097,079, titled “STUCK INSTRUMENT MANAGEMENT”, which issued on Sep. 24, 2024, which is hereby incorporated by reference in its entirety herein.

Although the system 100 of FIG. 1 is presented in the context of a ureteroscopic procedure, it should be understood that the principles disclosed herein may be implemented in any type of endoscopic and/or percutaneous procedure.

The medical system 100 includes a robotic system 10 (e.g., mobile robotic cart) configured to engage with and/or control a medical instrument 40 (e.g., ureteroscope) to perform a direct-entry procedure on a patient 7 supported on a table 15. For example, with reference to FIG. 1, the direct entry of the scope 40 into the urinary tract of the patient 7 may be made via the urethra 65. It should be understood that the direct-entry instrument 40 may be any type of medical instrument, including an endoscope (such as a ureteroscope), catheter (such as a steerable or non-steerable catheter), nephroscopes, laparoscope, or other type of medical instrument.

The medical system 100 includes a control system 50 configured to interface with the robotic system 10, provide information regarding the procedure, and/or perform a variety of other operations. For example, the control system 50 includes one or more display(s) 56 configured to present certain information to assist the physician 5 and/or other technician(s) or individual(s). The system 100 may further include an electromagnetic (EM) field generator 18, which may be held by one or more of the robotic arms 12a of the robotic system 10 or may be a stand-alone device.

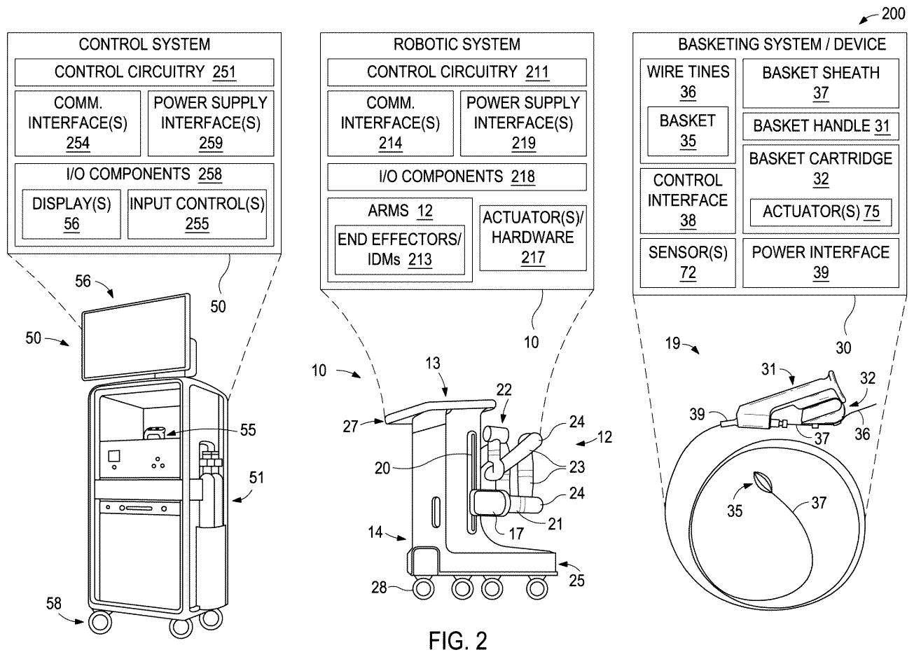

In an example use case, if the patient 7 has a kidney stone located in the kidney 70, the physician may execute a procedure to remove the stone through the urinary tract (63, 60, 65). In some embodiments, the physician 5 can interact with the control system 50 and/or the robotic system 10 to cause/control the robotic system 10 to advance and navigate the medical instrument 40 (e.g., a scope) from the urethra 65, through the bladder 60, up the ureter 63, and into the kidney 70 where the stone is located. The physician 5 can further interact with the control system 50 and/or the robotic system 10 to cause/control the advancement of a basketing device 30 (FIG. 2) through a working channel of the instrument 40, wherein the basketing device 30 (FIG. 2) is configured to facilitate capture and removal of a kidney stone.

The medical instrument 40 (e.g., scope, directly-entry instrument, etc.) can be advanced into the kidney 70 through the urinary tract. Specifically, a ureteral access sheath 90 may be disposed within the urinary tract and advanced to an area near the kidney 70. The medical instrument 40 may be passed through the ureteral access sheath 90 to gain access to the internal anatomy of the kidney 70. Once at the site of the kidney stone, the medical instrument 40 can be used to channel/direct the basketing device 30 (FIG. 2) to the target location. Once the stone has been captured in the distal basket portion 35 of the basketing device 30 (FIG. 2), the utilized ureteral access path may be used to extract the kidney stone from the patient 7.

The various scope-type instruments disclosed herein, such as the scope 40 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

With reference to FIG. 1 and FIG. 2, which shows an example embodiment of the control system 50 of FIG. 1, the control system 50 can be configured to provide various functionality to assist in performing a medical procedure. In some embodiments, the control system 50 can be coupled to the robotic system 10 and operate in cooperation therewith to perform a medical procedure on the patient 7. For example, the control system 50 can communicate with the robotic system 10 via a wireless or wired connection (e.g., to control the robotic system 10). Further, in some embodiments, the control system 50 can communicate with the robotic system 10 to receive position data therefrom relating to the position of the distal end of the scope 40, access sheath 90, or basketing device 30. Such positional data relating to the position of the scope 40, access sheath 90, or basketing device 30 may be derived using one or more electromagnetic sensors associated with the respective components. In some embodiments, the control system 50 can communicate with the EM field generator 18 to control generation of an EM field in an area around the patient 7.

With particular reference to FIG. 2, the robotic system 10 can be configured to at least partly facilitate execution of a medical procedure. The robotic system 10 can be arranged in a variety of ways depending on the particular procedure. The robotic system 10 can include one or more robotic arms 12 (12a, 12b, 12c) configured to engage with and/or control, for example, the scope 40 and/or the basketing system 30 to perform one or more aspects of a procedure. As shown, each robotic arm 12 can include multiple arm segments 23 coupled to joints 24, which can provide multiple degrees of movement/freedom. As shown in FIG. 1, the robotic system 10 is positioned proximate to the patient's legs and the robotic arms 12 are actuated to engage with and position the scope 40 for access into an access opening, such as the urethra 65 of the patient 7. When the robotic system 10 is properly positioned, the scope 40 can be inserted into the patient 7 robotically using the robotic arms 12b, manually by the physician 5, or a combination thereof. A scope-driver instrument coupling or “scope driver” 11 (i.e., instrument device manipulator (IDM)) can be attached to the distal portion of one of the arms 12b to facilitate robotic control/advancement of the scope 40. Another one of the arms 12c has associated therewith an instrument coupling/manipulator 19 configured to facilitate advancement and operation of the basketing device 30. The scope 40 includes one or more working channels through which additional tools, such as lithotripters, basketing devices, forceps, etc., can be introduced into the treatment site.

The robotic system 10 can be coupled to any component of the medical system 100, such as to the control system 50, the table 15, the EM field generator 18, the scope 40, the basketing system 30, and/or any type of percutaneous-access instrument (e.g., needle, catheter, nephroscope, etc.). In some embodiments, the robotic system 10 is communicatively coupled to the control system 50. For example, the robotic system 10 may be configured to receive control signals from the control system 50 to perform certain operations, such as to position one or more of the robotic arms 12 in a particular manner, manipulate the scope 40, manipulate the basketing system 30, and so on. In response, the robotic system 10 can control, using certain control circuitry 211, actuators 217, and/or other components of the robotic system 10 to perform the operations.

With reference to FIG. 2, the robotic system 10 generally includes an elongated support structure 14 (also referred to as a “column”), a robotic system base 25, and a console 13 at the top of the column 14. The column 14 includes one or more arm supports 17 (also referred to as a “carriage”) for supporting the deployment of the one or more robotic arms 12 (three shown in FIG. 2). The arm support 17 includes individually-configurable arm mounts that rotate along a perpendicular axis to adjust the base of the robotic arms 12 for desired positioning relative to the patient.

The arm support 17 is configured to vertically translate along the column 14, and can be connected to the column 14 through slots 20 that are positioned on opposite sides of the column 14 to guide the vertical translation of the arm support 17. Vertical translation of the arm support 17 allows the robotic system 10 to adjust the reach of the robotic arms 12 to meet a variety of table heights, patient sizes, and physician preferences. Similarly, the individually-configurable arm mounts on the arm support 17 can allow the robotic arm base 21 of robotic arms 12 to be angled in a variety of configurations.

The robotic arms 12 generally include robotic arm bases 21 and end effectors 22, separated by a series of linking arm segments 23 that are connected by a series of joints 24, each joint including one or more independent actuators 217. Each actuator 217 comprises an independently-controllable motor. Each independently-controllable joint 24 can provide or represent an independent degree of freedom available to the robotic arm. The robotic system base 25 can include wheel-shaped casters 28 that allow for the robotic system to easily move around the operating room prior to a procedure.

Positioned at the upper end of column 14, the console 13 can provide both a user interface for receiving user input and a display screen 16 (or a dual-purpose device such as, for example, a touchscreen) to provide the physician/user with both pre-operative and intra-operative data. As shown, the console 13 can also include a handle 27 to assist with maneuvering and stabilizing robotic system 10.

The end effector 213 of each of the robotic arms 12 includes, or is configured to have coupled thereto, an instrument device manipulator (IDM), which is attached using a mechanism changer interface (MCI). In some embodiments, the IDM can be removed and replaced with a different type of IDM, for example, a first type 11 of IDM manipulates an endoscope, while a second type 19 of IDM manipulates a basketing device. Another type of IDM is configured to hold an electromagnetic field generator 18. An MCI can provide power and control interfaces. For example, the interfaces can include connectors to transfer pneumatic pressure, electrical power, electrical signals, and/or optical signals from the robotic arm 12 to the IDM. The IDMs 213 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the medical device manipulators 213 can be attached to respective ones of the robotic arms 212, wherein the robotic arms 212 are configured to insert or retract the respective coupled medical instruments into or out of the treatment site.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or catheter basketing system 30. The control system 50 can include one or more display devices 56 to provide various information regarding a procedure.

To facilitate the functionality of the control system 50, the control system can include various components or “subsystems”. For example, the control system 50 can include the control electronics/circuitry 251, as well as one or more power supplies/supply interfaces 259, pneumatic devices, optical sources, actuators, data storage devices, and/or communication interfaces 254. In some embodiments, the control system 50 is movable, while in other embodiments, the control system 50 is a substantially stationary system.

The control system 50, basketing system 30, and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore.

The basketing system 30 includes various hardware and control components. For example, as shown in FIG. 2, the basketing system 30 can include a basket 35 formed of one or more wire tines 36, such as four wire tines disposed within a basketing sheath 37 over a length thereof, wherein the tines project from a distal end of the sheath 37 to form the basket 35. The tines 36 further extend from the proximal end of the sheath 37. The tines 36 may be configured to be slidable within the basketing sheath 37, subject to some amount of frictional resistance. The tines 36 and the sheath 37 can be coupled to respective actuators 75 of a basket cartridge component 32. The basket cartridge 32 may be physically and/or communicatively coupled to a handle portion/component 31 of the basketing system 30. The handle component 31 can be configured to be used to assist in basketing control either manually or through robotic control.

The basketing system 30 can be powered through a power interface 39 and/or controlled through a control interface 38, each or both of which may interface with a robotic arm/component of the robotic system 10. The basketing system 30 further includes one or more sensors 72, such as pressure and/or other force-reading sensors, which are configured to generate signals indicating forces experienced at/by one or more of the actuators 75 and/or other couplings of the basketing system 30. In some embodiments, the sensor(s) 72 include one or more sensors configured to directly measure forces are at or near the basket portion 35 of the tines 36. For example, a force sensor on the tip of the basket 35 and/or at a tip of an access sheath through which the basketing device 30 accesses the target anatomy can be used to directly detect forces on the basket 35 that result from the basket 35 becoming stuck on anatomy or on an opening at an end of the access sheath.

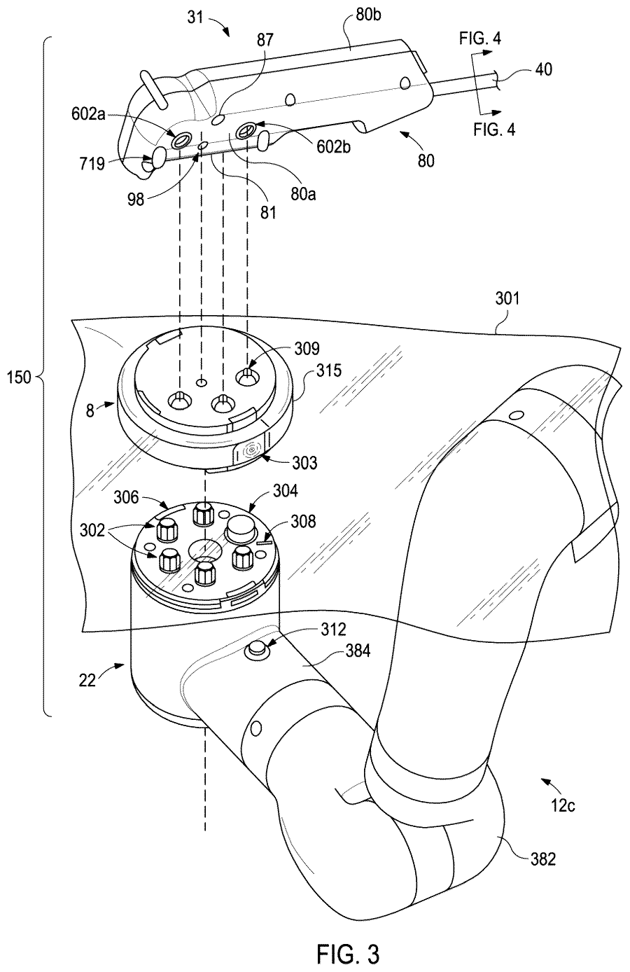

FIG. 3 shows an exploded view of an example instrument device manipulator assembly 150 associated with the robotic arm 12c in accordance with one or more embodiments. The instrument device manipulator assembly 150 (hereafter the “assembly 150”) includes an end effector 22 associated with a distal end of the robotic arm 12c, and further includes an instrument housing or “handle” 31 of a shaft-type instrument or instrument coupling/manipulator 19. The instrument handle 31 can incorporate mechanical (and/or electrical) means for rolling/rotating a shaft component associated therewith, such as an endoscope or other shaft-type instrument.

In some embodiments, the assembly 150 further includes an adapter component 8 that is mountable to the end effector 22 and configured to provide a driver interface between the end effector 22 and the instrument handle 31. The adapter 8 and/or the instrument handle 31 may be removable or detachable from the robotic arm 12c and may be devoid of any electro-mechanical components, such as motors, in some embodiments. This dichotomy may be driven by the need to sterilize medical instruments used in medical procedures and the inability to adequately sterilize expensive capital equipment due to their intricate mechanical assemblies and sensitive electronics. Accordingly, the instrument handle 31 and/or adapter 8 may be designed to be detached, removed, and interchanged from the end effector 22 (and thus the system) for individual sterilization or disposal. In contrast, the end effector 22 need not be changed or sterilized in some cases and may be draped using drape 301 for protection. The drape 301, alternately referred to as a “terile adapter,” may comprise a plastic sheet or the like, and may be disposed between the end effector 22 and the adapter 8 to provide a sterile barrier between the robot arm 12c and the instrument handle 31.

Additional information regarding the adapter 8 can be found in U.S. Pat. Pub. 2023/0135444, titled “INSTRUMENT ROLL CONTROL”, which published on May 4, 2023, which is hereby incorporated by reference in its entirety herein.

In some embodiments, the adapter 8 can include connectors to transfer pneumatic pressure, electrical power, electrical signals, and/or optical signals from the robotic arm 12c and/or end effector 22 to the instrument handle 31. The robotic arm 12c can advance or retract the coupled instrument handle 31 towards or away from the treatment site. The end effector 22 of the robotic arm 12c can include various components/elements configured to connect to and/or align with components of the adapter 8, instrument handle 31, and/or scope 40. For example, the end effector 22 can include drive outputs 302 (e.g., drive splines, gears, or rotatable disks with engagement features) to control/articulate a medical instrument, a reader 304 to read data from a medical instrument (e.g., radio-frequency identification (RFID) reader to read a serial number from a medical instrument), one or more fasteners 306 to attach the instrument handle 31 and/or adapter 8 to the end effector 22, marker(s) 308 to align with an instrument that is manually attached to a patient (e.g., access sheath) and/or to define a front surface of the assembly 150. In some embodiments, a portion (e.g., plate) 315 of the adapter 8 can be configured to rotate/spin independently of one or more other components of the adapter 8 and/or end effector 22 when coupled to the end effector 22. The adapter 8 can include one or more outputs 309 configured to mate/couple with a corresponding one or more input(s) 602a, 602b of the handle 31.

The instrument handle 31 includes a housing 80 that includes a base 80a and a cover 80b removably coupled to the base 80a to enclose various internal components of the instrument handle 31. The instrument handle 31 may further include a plurality of drive inputs 602a, 602b, 87 extending through the base 80a. In the illustrated embodiment, the instrument handle 31 includes three drive inputs 602a, 602b, 87, although other numbers of drive inputs can be included in other embodiments. The drive inputs 602a, 602b, 87 can be in fixed positions spaced apart along the base 80a of the instrument handle 31, which facilitates coupling the drive inputs 602a, 602b, 87 to the corresponding drive outputs 302 of the end effector 22, which may be in fixed positions spaced apart along a corresponding mating surface designed for modular use and attachment to a variety of other instruments. The handle 31 can include latching clips 719 or other latching features/means for physically coupling to corresponding structure of the adapter 8 and/or end effector 22.

An assembly within the instrument handle 31, described in more detail below, allows the drive inputs 602a, 602b to be used to drive articulation of the scope 40, whereas the drive input 87 can be used to drive roll of the shaft 40. Each of the drive inputs 602a, 602b, 87 can be operatively coupled to a corresponding drive output 302 on the end effector 22. For example, each drive input can comprise a receptacle configured to mate with a drive output that is configured as a spline. The drive inputs and drive outputs can be configured to engage to transfer motion therebetween. Thus, the drive outputs can be rotated to cause corresponding rotation of the drive inputs to control various functionality of the instrument handle 31.

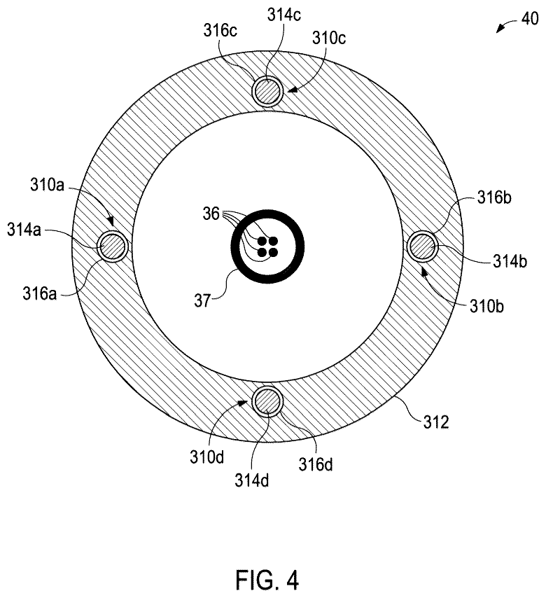

FIG. 4 shows a cross-sectional end view of the scope 40 of the instrument handle 31 as taken along the lines shown in FIG. 3, according to at least one aspect of the present disclosure. The scope 40 includes a sidewall 312 that defines one or more lumens 310a, 310b, 310c, 310d spaced angularly apart (equidistantly or non-equidistantly) around the sidewall 312 of the scope 40. The scope 40 also includes one or more pull wires 314a, 314b, 314c, 314d slidably disposed within a corresponding one of the lumens 310a-d. The pull wires 314a-d can include one or more cables, fibers, strings, and/or flexible shafts and can be made of any suitable or desirable materials, such as metallic and non-metallic materials, including stainless steel, Kevlar, tungsten, carbon fiber, and the like. Although a particular number of lumens 310a-d and pull wires 314a-d are illustrated in the figures, any number of lumens and/or pull wires can be implemented.

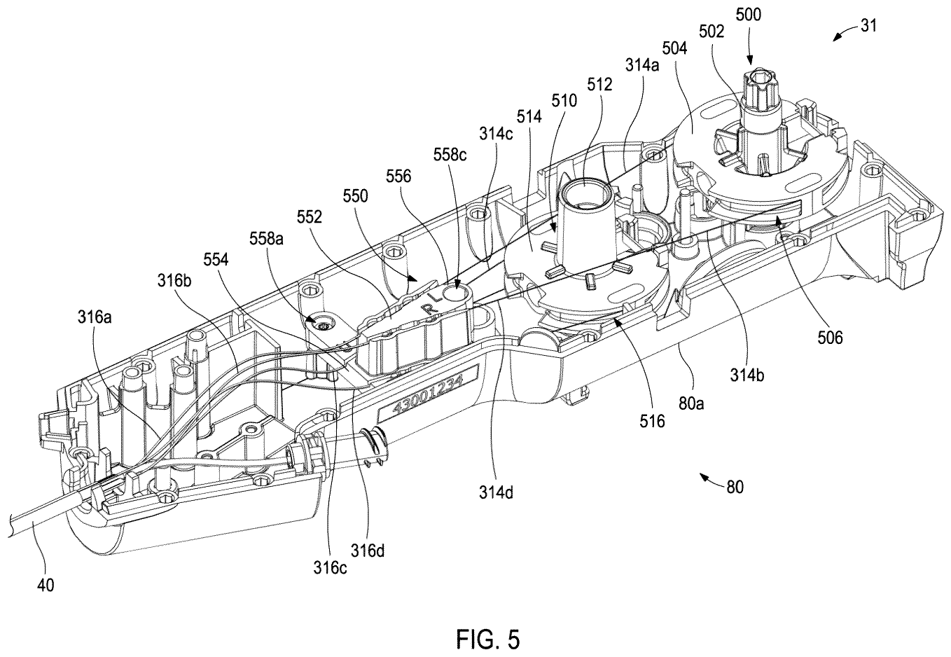

The pull wires 314a-d extend to the distal end of the scope 40. At a proximal side, the pull wires 314a-d are operatively coupled to articulation drives 500, 510 (FIG. 5) mounted in the housing 80 of the instrument handle 31. The articulation drives are configured to control articulation of the scope 40, as will be described in more detail below.

The scope 40 further includes a force isolation tube 316a, 316b, 316c, 316d disposed within each lumen 310a-d to radially interpose the corresponding pull wire 314a-d and inner wall of the lumen 310a-d. The force isolation tubes 316a-d, alternately referred to as “Bowden” tubes, serve as guides for the pull wires 314a-d as they axially move to articulate the scope 40. The force isolation tubes 316a-d are made of any suitable material, such as metal (e.g., stainless steel), and protect the sidewall 312 from frictional wear as the pull wires 314a-d move axially within the lumens 310a-d. In some embodiments, the sidewall 312 has a first stiffness and the force isolation tubes 316a-d have a second stiffness greater than the first stiffness.

FIG. 5 shows an enlarged isometric view of the interior of the instrument handle 31, in accordance with at least one aspect of the present disclosure. The cover 80b (FIG. 3) of the housing 80 is omitted to allow viewing of the various internal components of the instrument handle 31.

As illustrated, the instrument handle 31 may include a first articulation drive 500 and a second articulation drive 510 that are configured to control articulation of the scope 40. More specifically, the first articulation drive 500 may include a capstan 502 and a pulley 504 circumferentially disposed (positioned) therearound, and coupled to, the capstan 502. The capstan 502 may include or otherwise extend from the first drive input 602a (FIG. 3) such that rotation (actuation) of the drive input 602a correspondingly causes rotation of the capstan 502 and the pulley 504.

The pulley 504 may define a groove 506 to receive pull wires 314a, 314b. The pull wires 314a, 314b may be operatively coupled (e.g. directly or indirectly by way of one or more intervening components) to the pulley 504 such that rotation of the pulley 504 causes corresponding axial translation (movement) of the pull wires 314a, 314b. More specifically, rotation of the pulley 504 in a first radial direction (e.g. clockwise, as viewed in FIG. 5) causes the pulley 504 to apply tension to (pull) the pull wire 314a and release tension in (push to allow slack) the pull wire 314b, thereby causing the scope 40 to articulate in a first direction (e.g. right). Similarly, rotation of the pulley 504 in a second radial direction opposite the first radial direction (e.g. counterclockwise, as viewed in FIG. 5), causes the pulley 504 to apply tension to (pull) the pull wire 314b and release tension in (push to allow slack) the pull wire 314a, thereby causing the scope 40 to articulate in a second direction opposite the first direction (e.g. left).

Similar to the first articulation drive 500, the second articulation drive 510 may include a capstan 512 and a pulley 514 circumferentially disposed (positioned) around, and coupled to, the capstan 512. The capstan 512 may include or otherwise extend from the second drive input 602b (FIG. 3) such that rotation (actuation) of the drive input 602b correspondingly causes rotation of the capstan 512 and the pulley 514.

The pulley 514 may define a groove 516 to receive pull wires 314c, 314d. The pull wires 314c, 314d may be operatively coupled to (e.g. directly or indirectly by way of one or more intervening components) the pulley 514 such that rotation of the pulley 504 causes corresponding axial translation (movement) of the pull wires 314c, 314d. More specifically, rotation of the pulley 514 in a first radial direction (e.g. clockwise, as viewed in FIG. 5), causes the pulley 514 to apply tension to (pull) the pull wire 314c and release tension in (push to allow slack) the pull wire 314d, thereby causing the scope 40 to articulate in a third direction (e.g. up). Similarly, rotation of the pulley 514 in a second radial direction opposite the first radial direction (e.g. counterclockwise, as viewed in FIG. 5), causes the pulley 514 to apply tension to (pull) the pull wire 314d and release tension in (push to allow slack) the pull wire 314c, thereby causing the scope 40 to articulate in a fourth direction opposite the third direction (e.g. down).

### Systems and Methods for Bowden Tube Termination

As discussed above, the pull wires 314a-d may extend through corresponding force isolation tubes 316a-d, and the pull wires 314a-d and the corresponding force isolation tubes 316a-d are jointly received within the sidewall of the scope 40. Routing the pull wires 314a-d and the force isolation tubes 316a-d within a conventional housing is typically difficult because the pull wires 314a-d exhibit a very small gauge and must be fed through small holes. This process often requires the use of a microscope to accurately place and feed the pull wires 314a-d.

According to embodiments of the present disclosure, the instrument handle 31 further includes a terminator piece or “terminator” 550 configured to ease installation of the pull wires 314a-d and force isolation tubes 316a-d within the housing 80. In some embodiments, the terminator 550 may comprise an integral component to the housing 80. In such embodiments, the terminator 550 may be constructed (e.g. molded, cast, 3D printed) as a unitary piece with the base 80a. In other embodiments, however, the terminator 550 may comprise a separate component part that is removably coupled to the interior of the housing 80.

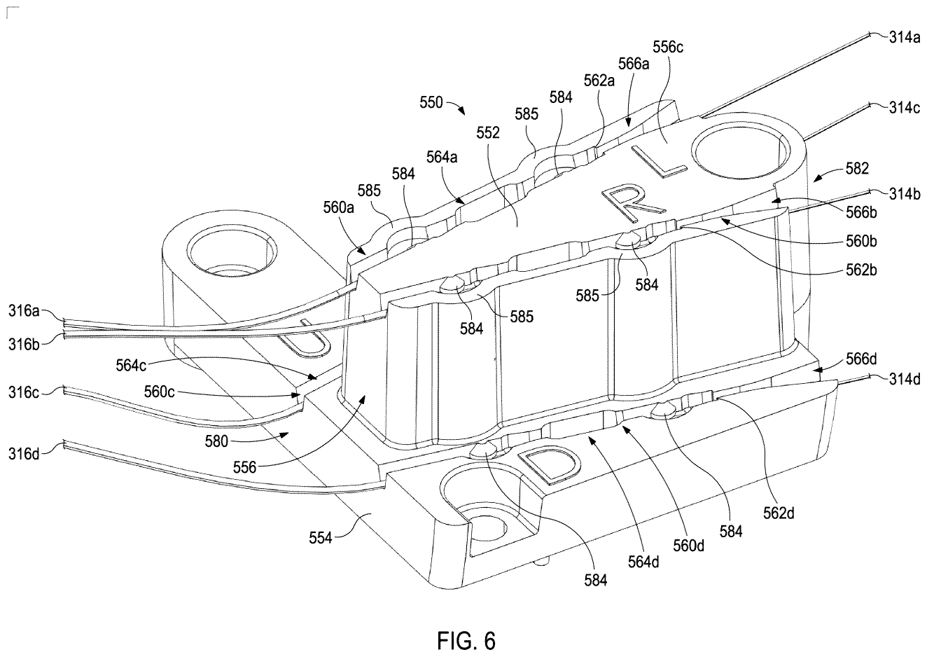

The terminator 550 provides a body 552 that includes a base 554 and a column 556 extending from the base 554. The base 554 may define a first aperture 558a on a first lateral side of the column 556 and a second aperture 558b (FIG. 6) on a second lateral side of the column 556. The column 556 may further define a third aperture 558c. The apertures 558a-c may be sized to receive corresponding fasteners (e.g. a bolt, screw, rivet, etc.) to removably couple the terminator 550 to the housing 80.

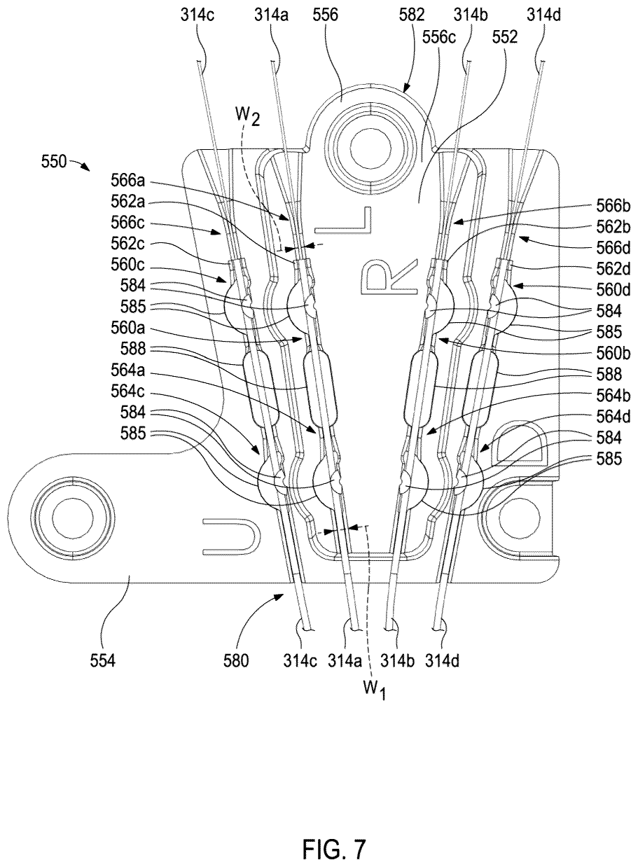

FIG. 6 is an enlarged, isometric view of the terminator 550, and FIG. 7 provides a top-down view of FIG. 6, in accordance with at least one aspect of the present disclosure. With reference to FIGS. 6 and 7, the body 552 defines or otherwise provides a plurality of elongate wire grooves 560a, 560b, 560c, 560d sized to receive the pull wires 314a-d and at least of a portion of a corresponding force isolation tube 316a-d. In some embodiments, one or more of the wire grooves 560a-d may be U-shaped and may exhibit a variable width. In other embodiments, however, other shapes and geometries may define the wire grooves 560a-d, without departing from the scope of the disclosure.

As illustrated, first and second wire grooves 560a and 560b may be defined in the column 556, such as being defined into a top surface 556c thereof. Moreover, third and fourth wire grooves 560c and 560d may be defined in the base 554. More specifically, the third wire groove 560c may be defined on a first lateral side of the column 556, and the fourth wire groove 560d may be defined on a second lateral side of the column 556. In some embodiments, the first and third wire grooves 560a,c may extend substantially parallel to one another and the second and fourth wire grooves 560b,d may extend substantially parallel to one another. In such embodiments, the first and third wire grooves 560a,c may be angled relative to the second and fourth wire grooves 560b,d such that a non-zero angle is defined therebetween, such as 5°, 10°, 15°, or 20°, as examples. Accordingly, the first and second wire grooves 560a,b may diverge (or converge) relative to each other, and the third and fourth wire grooves 560c,d may diverge (or converge) relative to each other.

A shoulder 562a, 562b, 562c, 562d is defined in each wire groove 560a-d, and each shoulder 562a-d provides a point of separation in the corresponding wire groove 560a-d to separate the wire groove 560a-d into corresponding first wire groove portions 564a, 564b, 564c, 564d and corresponding second wire groove portions 566a, 566b, 566c, 566d. The first wire groove portions 564a-d extend from the corresponding shoulder 562a-d toward a forward end 580 of the terminator 550 (e.g., distal to) and the opposing sidewalls thereof exhibit a first width w₁. In contrast, the second wire groove portions 566a-d extend from the corresponding shoulder 562a-d toward a rearward end 582 of the terminator 550 (e.g., proximal to) and the opposing sidewalls thereof exhibit a second width w₂ that is different (less) than the first width w₁.

The pull wires 314a-d each exhibit a third width (gauge) that is smaller than the first and second widths w₁, w₂ such that the pull wires 314a-d are able to extend through the first and second wire groove portions 564a-d, 566a-d between the forward and rearward ends 580, 582 to be received at the pulleys 504, 514 (FIG. 5). The force isolation tubes 316a-d may exhibit a fourth width (gauge) that is less than the first width w₁ but greater than the second width w₂. Accordingly, the force isolation tubes 316a-d may be able to extend within the first wire groove portions 564a-d, but are prevented from entering the second wire groove portions 566a-d due to the shoulder 526a-d. Due to their reduced diameter relative to the force isolation tubes 316a-d, the shoulders 562a-d may apply a resistive longitudinal (axial) force to the force isolation tubes 316a-d should the force isolation tubes 316a-d be drawn rearwardly toward the pulleys 504, 514 (FIG. 5), such as during articulation of the scope 40 by way of the pull wires 314a-d.

The body 552 may further define a plurality of tabs 584 that allow the pull wires 314a-d to be threaded (e.g., vertically inserted) into the wire grooves 560a-d, while substantially preventing the pull wires 314a-d and the force isolation tubes 316a-d from exiting the wire grooves 560a-d vertically while tension is applied. Two tabs 584 may be associated with each wire groove 560a-d (i.e. eight total), and each tab 584 may extend from one of the sidewalls of the first wire groove portion 564a-d toward an opposing sidewall. In at least one embodiment, the opposing sidewall may comprise an arcuate surface 585, thereby resulting in an arcuate gap defined between the tab 584 and the corresponding arcuate surface 585. While two tabs 584 are provided in each wire groove 560a-d, the terminator 550 may include more than two tabs (e.g. three, four, or five) or less than two tabs (e.g. one) in each wire groove 560a-d.

The gaps allow the pull wires 314a-d to bypass the tabs 584 and enter or exit the wire grooves 560a-d vertically when tension is removed, but prevents the pull wires 314a-d and the force isolation tubes 316a-d from bypassing the tabs 584 and thereby escaping the wire grooves 560a-d when tension is applied. As discussed in more detail below, the tabs 584 require that the force isolation tubes 316a-d be inserted into the wire grooves 560a-d from the forward end 580.

A pocket or recess 588 (FIG. 7) may be defined in the first wire groove portions 564a-d and configured to be filled with an adhesive (e.g. glue) to adhere the force isolation tubes 316a-d within the corresponding wire grooves 560a-d. The adhesive in the pockets 588 and the tabs 584 may co-operatively function to maintain the axial (and vertical) position of the force isolation tubes 316a-d relative to the terminator 550. For example, the tabs 584 prevent the force isolation tubes 316a-d from exiting the terminator 550 vertically, and the adhesive prevents the force isolation tubes 316a-d from exiting the terminator 550 along the longitudinal length of the corresponding wire groove 560a-d.

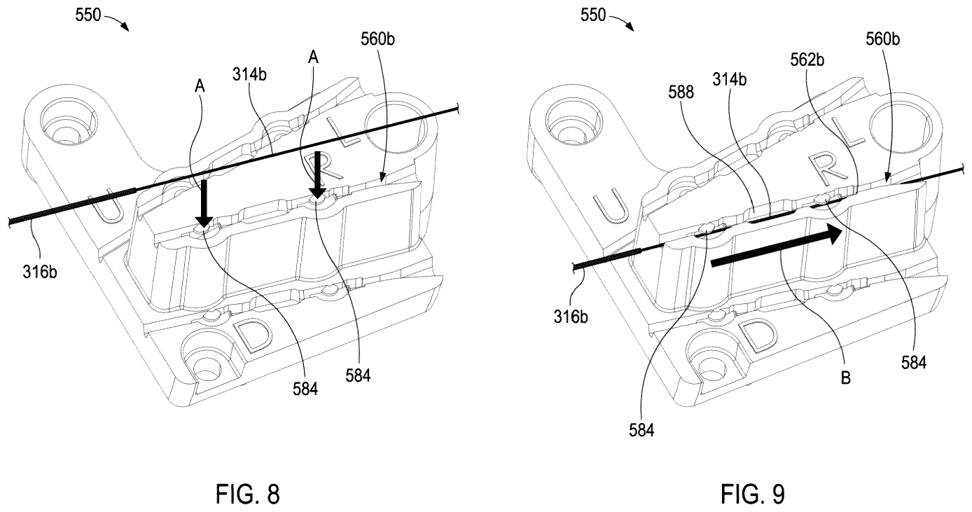

FIGS. 8 and 9 provide an illustrative example of assembling the pull wire 314b and the corresponding force isolation tube 316b into the wire groove 560b of the terminator 550, in accordance with at least one aspect of the present disclosure. As shown in FIG. 8, the pull wire 314b is first inserted in a first direction (e.g. vertically downward), as indicated by the arrows A, bypassing the tabs 584, and into the first and second wire groove portions 564b, 566b of the wire groove 560b. Without tension applied on the pull wire 314b (or with a small amount), the pull wire 314b can be threaded into the wire groove 560b vertically. Once the pull wire 314b is positioned in the wire groove 560b, the force isolation tube 316b may be advanced along the pull wire 314b in a second direction, as indicated by the arrow B, transverse (orthogonal) to the first direction (e.g. longitudinal). The force isolation tube 316b may slide along the pull wire 314b and under (beneath) the tabs 584 until contacting (abutting) the shoulder 562b of the wire groove 560b. The force isolation tube 560b may also be adhered to the terminator 550 using an adhesive in the pocket 588.

While the foregoing discussion was directed to assembling the pull wire 314b and the force isolation tube 316b into the wire groove 560b, it should be understood that the other pull wires 314a,c,d, and force isolation tubes 316a,c,d would be assembled into their respective wire grooves 560a,c,d in a similar manner.

### Systems and Methods for Mounting a Cable and Circuit Board

As discussed elsewhere herein, the scope 40 (FIG. 1) of the system 100 (FIG. 1) can be configured to navigate within the human anatomy, such as within a natural orifice or lumen of the human anatomy. The distal end of the scope 40 may include a camera (not shown) for viewing various organs, cavities, lumens, chambers, or spaces of a body, such as the urinary tract, the kidneys, the bronchus, the colon, a joint, the bladder, the rectum, and so on. The camera is typically in electrical communication with an external source to receive power therefrom and output a video signal thereto. For example, the camera may be in electrical communication with the control system 10 (FIG. 1) via a circuit board mounted in the housing 80 (FIG. 3) of the instrument handle 31 (FIG. 3) and a cable that extends from the circuit board to the external source. This cable is often subjected to external forces, such as handling loads, yanking, or tripping. Accordingly, systems and methods for securing the cable to the instrument handle 31 (FIG. 1) to withstand these external forces without failure are desirable.

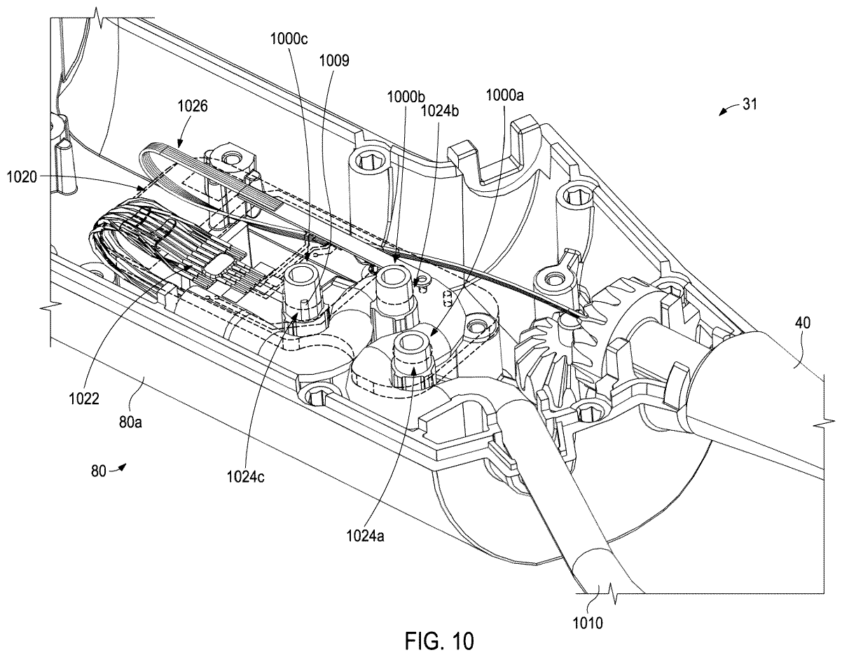

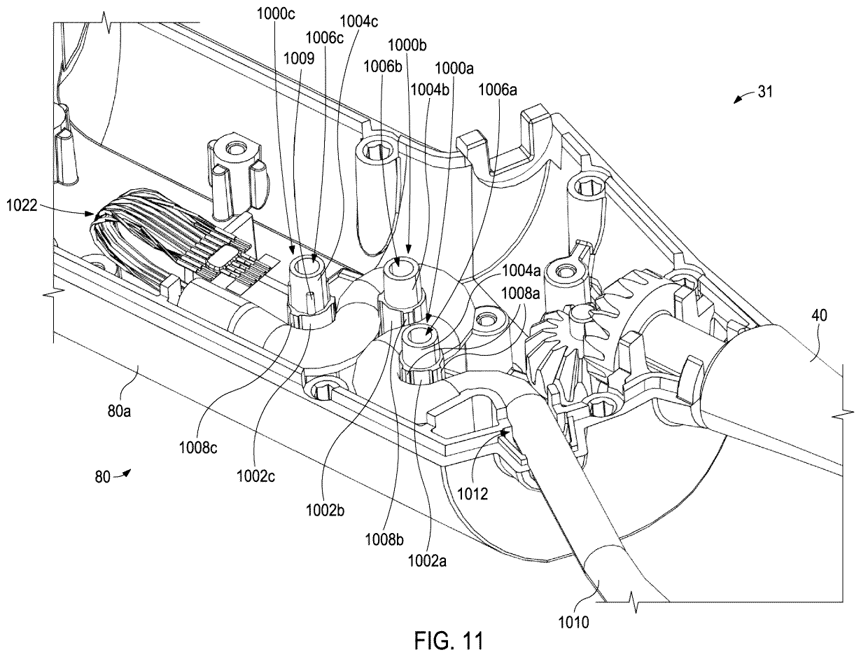

FIGS. 10 and 11 show an isometric view of the instrument handle 31, in accordance with at least one aspect of the present disclosure. In FIGS. 10 and 11, the cover 80b of the housing 80 is omitted to allow viewing of the various internal components of the instrument handle 31. Various internal components within the instrument handle 31 are omitted to allow for improved viewing of the below-described components. In FIG. 11, various additional component parts are omitted to allow for better viewing of other components.

With reference first to FIG. 11, the base 80a of the housing 80 may provide or otherwise define a first post 1000a, a second post 1000b, and a third post 1000c that may be offset from each other and otherwise arranged in a triangular juxtaposition. The first post 1000a may be separated from the second post 1000b by a first distance to define a first gap therebetween and the second post 1002b may be separated from the third post 1000c by a second distance which could be the same, or similar, to the first distance, to define a second gap therebetween. While three posts are shown and described, the housing 80 may include less than three posts (e.g. two posts) or more than three posts (e.g. four, five, or six posts).

Each post 1000a-b may comprise a first post portion 1002a, 1002b, 1002c that extends from the base 80a and a second post portion 1004a, 1004b, 1004c that extends from the corresponding first post portion 1000a-c. The first post portions 1002a-c may define a first diameter and the second post portions 1004a-c may define a second diameter different (less) than the first diameter. The second post portions 1004a-c may each define an aperture or recess 1006a, 1006b, 1006c sized to receive a respective post (not shown) extending from the cover 80b (FIG. 3), which may be helpful in removably attaching the cover 80b (FIG. 3) to the base 80a.

The first post portions 1002a-c may each define, or otherwise provide, gripping features 1008a, 1008b, 1008c circumferentially disposed therearound. The second post portion 1004c of the third post 1000c may define, or otherwise provide, a rib 1009. While only one rib 1009 is illustrated, the second post portion 1004c may provide, otherwise define, more than one rib 1009 (e.g. two, three, or four ribs) circumferentially disposed therearound. While only the second post portion 1004c of the third post 1000c of the illustrated embodiment provides a rib 1009, other embodiments are envisioned in which the second post portions 1004a,b of the first and second posts 1000a,b also provide a rib, like rib 1009.

As referenced above, the camera of the scope 40 is typically in electrical communication with an external source, such as the control system 10 (FIG. 1), via a cable that extends from the external source. As shown in FIG. 10, a cable 1010 is provided that may enter the housing 80 via an aperture 1012 defined in the base 80a.

The posts 1000a-c may help co-operatively define a torturous path through the housing 80 through which the cable 1010 may be routed. For instance, the torturous path may include a partial wrap, or turn, around the first post 1000a in a first radial direction (e.g. clockwise, as viewed in FIG. 11), an extension of the cable 1010 into the first gap between the first and second posts 1000a,b, a partial wrap, or turn, around the second post 1000b in a second radial direction opposite the first radial direction (e.g. counterclockwise, as viewed in FIG. 11), an extension of the cable into the second gap between the second and third posts 1000b,c, and a partial wrap, or turn, around the third post 1000c in the first radial direction.

The above-describe torturous path is merely one example of how the cable 1010 can be routed through a torturous path defined by the posts 1000a-c. Another example may include, for example, a partial wrap, or turn, around the second post 1000b in a first radial direction (e.g. counterclockwise, as viewed in FIG. 11), an extension of the cable 1010 into the first gap between the first and second posts 1000a,b, a partial wrap, or turn, around the first post 1000b in a second radial direction opposite the first radial direction (e.g. clockwise, as viewed in FIG. 11), an extension of the cable into the second gap between the second and third posts 1000b,c, and a partial wrap, or turn, around the third post 1000c in the first radial direction.

The cable 1010 may define a diameter that is the same, or similar, to the first distance between the first and second posts 1000a,b and the second distance between the second and third posts 1000b,c. Accordingly, when positioned in the torturous path, the cable 1010 may frictionally engage the first post portions 1002a-c of the posts 1000a-c. Furthermore, the gripping features 1008a-c on the first post portions 1002a-c may frictionally engage the cable 1010 while positioned in the torturous path to enhance the frictional engagement between the cable 1010 and the posts 1000a-c.

The tight looping of the tortuous path provides an improved frictional grip on the cable 1010 to maintain a position thereof, with the frictional grip by the posts 1000a-c on the cable 1010 increasing when an external force (e.g. handling loads, yanking, tripping, etc.) is applied to the cable 1010. Accordingly, the torturous path of the posts 1000a-c may maintain a position of the cable 1010 without requiring fasteners to directly mount the cable 1010 to the base 80a.

Referring now to both FIGS. 10 and 11, once the cable 1010 is properly routed through the torturous path, as generally described above, leads (connectors) 1022 of the cable 1010 may be folded to position a circuit board 1020 (shown in phantom) offset (vertically above) from the cable 1020. An electrical connector 1026 may be electrically coupled to the circuit board 1020 and extend through the scope 40 to electrically coupled with the camera.

The circuit board 1020 may define a first aperture 1024a sized to receive the first post 1000a, a second aperture 1024b sized to receive the second post 1000b, and a third aperture 1024c sized to receive the third post 1000c. More specifically, the first, second, and third apertures 1024a-c may include a diameter that is the same, or substantially similar to the second diameter defined by the second post portions 1004a-c.

Once the circuit board 1020 is mounted to the housing 80 by receiving the posts 1000a-c in the corresponding apertures 1024a-c, the cable 1010 may be sandwiched between the circuit board 1020 and the base 80a, which may further maintain a position of the cable 1010 within the housing 80. In addition, as the posts 1000a-c are received at the corresponding apertures 1024a-c, the rib 1009 may engage the circuit board 1020 in the aperture 1024c, as shown in FIG. 10, to generate a friction fit therebetween (alternatively referred to as a press fit or interference fit). Accordingly, the frictional engagement between the circuit board 1020 and the rib 1009 may maintain a position of the circuit board 1020 relative to the housing 80, which may in turn maintain a position of the cable 1010 sandwiched between the circuit board 102 and the base 80a.

Embodiments disclosed herein include:

A. A surgical instrument comprising a housing, a scope extending from the housing, a terminator arranged within the housing and including a body that provides a wire groove and a shoulder defined in the wire groove, a force isolation tube extending from the scope and being received within the wire groove to the shoulder, a pull wire extending within the force isolation tube and through the wire groove, and a pulley arranged within the housing to receive the pull wire from the terminator, wherein rotation of the pulley acts on the pull wire to articulate the scope.

B. A method comprising inserting a pull wire into a wire groove of a terminator arranged within a housing of a surgical instrument in a first direction and sliding a force isolation tube along the pull wire in a second direction transverse the first direction and toward a shoulder defined within the wire groove.

C. A surgical instrument comprising a housing that provides a first post and a second post, a torturous pathway defined in the housing and extending at least partially around the first and second posts, a cable extending along the torturous path, and a circuit board electrically coupled to the cable, wherein the circuit board defines an aperture to receive the first post, thereby sandwiching the cable between the housing and the circuit board.

D. A surgical instrument comprising a housing providing a first post and a second post, a torturous path defined in the housing and extending at least partially around the first and second posts, a cable extending along the torturous path, and a circuit board electrically coupled to the cable, wherein the circuit board defines an aperture to receive the first post, wherein the first post frictionally engages the circuit board to maintain a position of the circuit board and the cable based on the aperture receiving the first post.

E. A method comprising routing a cable through a torturous path defined by a first post and a second post within a housing of a surgical instrument, positioning the first post through an aperture defined in a circuit board that is electrically coupled to the cable, thereby sandwiching the cable between the housing and the circuit board, and press fitting the circuit board with the first post.

Each of embodiments A-E may have one or more of the following additional elements in any combination: Element 1: wherein the shoulder prevents the force isolation tube from longitudinally extending through the wire groove to the pulley. Element 2: wherein the pull wire defines a first width, the force isolation tube defines a second width greater than the first width, the wire groove defines a third width greater than the first and second widths, and the shoulder defines a fourth width in the wire groove that is greater that the first width, but less than the second width. Element 3: wherein the body further defines a tab that extends into the wire groove to prevent the force isolation tube from bypassing the tab to enter or exit the wire groove. Element 4: wherein the tab allows the pull wire to bypass the tab to enter or exit the wire groove. Element 5: wherein the tab extends from a first sidewall of the wire groove toward an arcuate surface of the wire groove, thereby defining an arcuate gap between the tab and the arcuate surface. Element 6: wherein a pocket is defined in the wire groove, and wherein an adhesive applied to the pocket adheres the force isolation tube to the terminator. Element 7: wherein the terminator is removably coupled to the housing. Element 8: wherein the body further defines an aperture to receive a fastener therethrough to removably couple the terminator to the housing. Element 9: wherein the terminator forms an integral part of the housing. Element 10: wherein the pull wire is a first pull wire, and the wire groove is a first wire groove, and wherein the body further provides a second wire groove configured to receive the second pull wire. Element 11: wherein the first and second wire grooves diverge from one another. Element 12: wherein the body comprises a base, and the first wire groove is defined in the base and a column extending from the base, wherein the second wire groove is defined in the column. Element 13: further comprising receiving the pull wire from the terminator at a pulley rotatably mounted within the housing, rotating the pulley and thereby acting on the pull wire, and applying, with the shoulder, a resistive force to the force isolation tube as the pulley rotates. Element 14: wherein inserting the pull wire into the wire groove comprises bypassing a tab provided by the terminator and extending into the wire groove. Element 15: wherein sliding the force isolation tube comprises sliding the force isolation tube under the tab. Element 16: wherein bypassing the tab comprises bypassing the tab through an arcuate gap defined between the tab and an arcuate surface of the wire groove that opposes the tab. Element 17: further comprising adhering the force isolation tube to the terminator. Element 18: further comprising inserting a second pull wire into a second wire groove of the terminator that is angled relative to the first wire groove. Element 19: wherein the first post provides a rib that engages the circuit board to form an interference fit between the post and the circuit board. Element 20: wherein the torturous path includes a turn at the first post, an extension between the first and second posts, and a turn at the second post. Element 21: wherein the surgical instrument lacks a fastener to directly secure the circuit board or the cable to the housing. Element 22: wherein the surgical instrument further comprises a scope extending from the housing. Element 23: wherein the scope includes a camera in electrical communication with the circuit board. Element 24: further comprising a wire operably coupled to the scope and a pulley operably coupled to the wire, wherein rotation of the pulley pulls the wire to articulate the scope. Element 25: wherein the first post provides a rib that frictionally engages the circuit board to maintain the position of the circuit board and the cable. Element 26: wherein the torturous path includes a turn at the first post, an extension between the first and second posts, and a turn at the second post. Element 27: wherein the surgical instrument lacks a fastener to directly secure the circuit board or the cable to the housing. Element 28: wherein the surgical instrument further comprises a scope extending from the housing. Element 29: wherein the scope includes a camera in electrical communication with the circuit board. Element 30: further comprising a wire operably coupled to the scope and a pulley operably coupled to the wire, wherein rotation of the pulley pulls the wire to articulate the scope. Element 31: wherein press fitting the circuit board comprises pressing fitting the circuit board with a rib extending from the first post. Element 32: wherein routing the cable through the torturous path comprises at least partially wrapping the cable around the first post, extending the cable between the first and second posts, and at least partially wrapping the cable around the second post. Element 33: wherein the method lacks directly securing the circuit board or the cable to the housing with a fastener. Element 34: wherein the surgical instrument further comprises a camera, and wherein the method further comprises electrically coupling the circuit board to the camera. Element 35: further comprising mounting the surgical instrument to robotic arm.

By way of non-limiting example, exemplary combinations applicable to A, B, C, D, and E include: Element 3 with Element 4; Element 3 with Element 5; Element 7 with Element 8; Element 10 with Element 11; Element 10 with Element 12; Element 14 with Element 15; Element 14 with Element 17; Element 22 with Element 23; Element 22 with Element 24; Element 28 with Element 29; Element 28 with Element 30.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical instrument, comprising:
a housing;
a scope extending from the housing;
a terminator arranged within the housing and including a body that provides a wire groove and a shoulder defined in the wire groove;
a force isolation tube extending from the scope and being received within the wire groove to the shoulder;
a pull wire extending within the force isolation tube and through the wire groove; and
a pulley arranged within the housing to receive the pull wire from the terminator,
wherein rotation of the pulley acts on the pull wire to articulate the scope.

2. The surgical instrument of claim 1, wherein the shoulder prevents the force isolation tube from longitudinally extending through the wire groove to the pulley.

3. The surgical instrument of claim 1, wherein:
the pull wire defines a first width;
the force isolation tube defines a second width greater than the first width;
the wire groove defines a third width greater than the first and second widths; and
the shoulder defines a fourth width in the wire groove that is greater that the first width, but less than the second width.

4. The surgical instrument of claim 1, wherein the body further defines a tab that extends into the wire groove to prevent the force isolation tube from bypassing the tab to enter or exit the wire groove.

5. The surgical instrument of claim 4, wherein the tab allows the pull wire to bypass the tab to enter or exit the wire groove.

6. The surgical instrument of claim 4, wherein the tab extends from a first sidewall of the wire groove toward an arcuate surface of the wire groove, thereby defining an arcuate gap between the tab and the arcuate surface.

7. The surgical instrument of claim 1, wherein a pocket is defined in the wire groove, and wherein an adhesive applied to the pocket adheres the force isolation tube to the terminator.

8. The surgical instrument of claim 1, wherein the terminator is removably coupled to the housing.

9. The surgical instrument of claim 8, wherein the body further defines an aperture to receive a fastener therethrough to removably couple the terminator to the housing.

10. The surgical instrument of claim 1, wherein the terminator forms an integral part of the housing.

11. The surgical instrument of claim 1, wherein the pull wire is a first pull wire, and the wire groove is a first wire groove, and wherein the body further provides a second wire groove configured to receive the second pull wire.

12. The surgical instrument of claim 11, wherein the first and second wire grooves diverge from one another.

13. The surgical instrument of claim 11, wherein the body comprises:
a base, and the first wire groove is defined in the base; and
a column extending from the base, wherein the second wire groove is defined in the column.

14. A method, comprising:
inserting a pull wire into a wire groove of a terminator arranged within a housing of a surgical instrument in a first direction; and
sliding a force isolation tube along the pull wire in a second direction transverse the first direction and toward a shoulder defined within the wire groove.

15. The method of claim 14, further comprising:
receiving the pull wire from the terminator at a pulley rotatably mounted within the housing;
rotating the pulley and thereby acting on the pull wire; and
applying, with the shoulder, a resistive force to the force isolation tube as the pulley rotates.

16. The method of claim 14, wherein inserting the pull wire into the wire groove comprises bypassing a tab provided by the terminator and extending into the wire groove.

17. The method of claim 16, wherein sliding the force isolation tube comprises sliding the force isolation tube under the tab.

18. The method of claim 16, wherein bypassing the tab comprises bypassing the tab through an arcuate gap defined between the tab and an arcuate surface of the wire groove that opposes the tab.

19. The method of claim 14, further comprising adhering the force isolation tube to the terminator.

20. The method of claim 14, further comprising inserting a second pull wire into a second wire groove of the terminator that is angled relative to the first wire groove.
